(19) United States
(12) Patent Application Publication
(10) Pub. No.: US 2002/0076443 A1
(43) Pub. Date: Jun. 20, 2002

(54) Multiple phase cross-linked compositions and uses thereof

(76) Inventors: Stanley Stein, East Brunswick, NJ (US); Bo Qiu, East Brunswick, NJ (US)

Correspondence Address: KLAUBER & JACKSON, 411 HACKENSACK AVENUE, HACKENSACK, NJ, 07601

(21) Appl. No.: 09/883,842

(22) Filed: Jun. 18, 2001

Related U.S. Application Data
(60) Provisional application No. 60/212,511, filed on Jun. 19, 2000.

(51) Int. Cl. A61K 9/14

(52) U.S. Cl. 424/486; 424/488

(57) ABSTRACT

The present invention is directed to pharmaceutical compositions, and method for preparing pharmaceutical compositions, comprising a cross-linked matrix physically entrapping at least one therapeutic agent. The matrix may comprise one or more phases in addition to an aqueous phase, such as a solid and/or oil phase. The matrix of the invention has at least one controlled release in-vivo kinetic profile, and may have additional profiles for the same agent. The matrix may also comprise more than one therapeutic agent, and each additional therapeutic agent may have one or more controlled release in-vivo kinetic profile.

### CROSS-REFERENCE TO RELATED APPLICATION

Priority under 35 U.S.C. §119(e) is claimed to Provisional Application Serial No. 60/212,511, filed Jun. 19, 2000, incorporated herein by reference in its entirety.

### FIELD OF THE INVENTION

The present invention relates to materials, methods for their preparation, and compositions including pharmaceutical compositions that comprise a cross-linked matrix comprising a polymer and multiple phases. Such multiple-phase matrices or compositions exhibit new and useful physical properties including stability of oil-water emulsions, and controlled release kinetic profiles of active agents contained therein, making them suitable for controlled release formulations of various agents such as therapeutic agents for uses including the prophylaxis or treatment of conditions and diseases.

### BACKGROUND OF THE INVENTION

Therapeutic agents with short half lives, such as most proteins, must be administered by injection at closely repeated intervals to maintain therapeutic benefit, since their in vivo half-lives are minutes to hours. A prominent approach for extending the half-life of a protein to a period of hours or days is to covalently append to it one or more chains of poly(ethylene glycol) (PEG). Appended PEG chains may provide the favorable pharmacologic properties of protection of the underlying protein from immune surveillance and proteolytic enzymes, in addition to the lower rate of clearance from the bloodstream (Davis, S., Abuchowski, A., Park, Y. K. and Davis, F. F. (1981) Clin. Exp. Immunol. 46, 649-652.). However, the successful use of this “pegylation” technology is highly and unpredictably dependent on both the particular protein and the conjugation chemistry, and is effective for a few days at most. It is also not directly suited to all short-lived therapeutic agents.

Another approach to extending the in vivo lifetime of a therapeutic agent is to administer that agent encapsulated in a sustained release depot. Protein encapsulation processes that require the use of organic solvents or heating potentially physically modify, i.e. denature, a protein drug. A process for preparing protein microparticles by heating in the presence of polymers is described by Woiszwillo et al. (U.S. Pat. No. 5,849,884). A process in which the protein drug is contacted with an organic solvent is described by Zale et al. (U.S. Pat. No. 5,716,644).

Encapsulation processes that require chemical bond formation among the encapsulation reagents might have reactions that unintentionally chemically modify the protein. Thus, this latter method is less favored, since for the example of proteins, which are typically composed of amino acids having a variety of side chain functional groups, chemical modification may impair the pharmacological activity. The same impairment may be imparted to other therapeutic agents.

It is toward the development of new controlled release delivery systems for small-molecule drugs, proteins and other therapeutic agents, particularly for those with short in-vivo lifetimes, that the present application is directed. Furthermore, the new and useful properties of such controlled release delivery materials have found uses beyond the pharmaceutical uses, in the handling, storage, and delivery of industrial agents.

The citation of any reference herein should not be construed as an admission that such reference is available as “Prior Art” to the instant application.

### SUMMARY OF THE INVENTION

In its broadest aspect, the present invention is directed to compositions and methods for preparing such compositions, the compositions being cross-linked polymer matrices comprising a homogeneous plurality of phases, one of which is an aqueous phase. The methods for preparing such compositions comprise preparing a homogeneous mixture of at least two phases, one of which is an aqueous phase, and at least one polymer capable of being cross-linked present in at least one of the phases, and forming cross-links between the polymer molecules. The phase other than the aqueous phase may be one or more oil (lipid) phases or one or more solid phases, or multiple different combinations of the phases, such as two solid phases each comprising a different agent in a single aqueous phase or in an emulsion of the aqueous and oil (lipid) phases, or an emulsion of two different oil (lipid) phases in a single aqueous phase. Preferably, the polymer is in the aqueous phase.

In a preferred embodiment, at least one active agent is present in at least one of the phases, such that the at least one agent is physically entrapped within the composition. One or more excipients may be included in the composition to aid in the formation, stability and/or release characteristics of the composition, such as a surfactant to aid in the formation of an emulsion, a polymeric counterion to aid in the insolubilization of a polymeric active agent within the composition, or a proteinase inhibitor to maintain the stability of a proteinaceous active agent within the matrix.

As noted above, an agent may be physically entrapped within one or more phases in the matrix of the invention. Such physical entrapment generally relates to and refers to the cross-linking of the polymer which non-covalently entraps the components of the composition, including any suspended (solid phase) material, the emulsion, or any additional phases present. The active agent need not necessarily be present in the same phase that the polymer is present, which as noted above is preferably the aqueous phase. A preferred embodiment comprises an aqueous and an oil (lipid) phase, with the polymer in the aqueous phase and the therapeutic agent entrapped within the polymer within the oil (lipid) phase or the aqueous phase.

In one embodiment, the at least one active agent is a therapeutic agent. The therapeutic agent may be a small organic molecule, nucleic acid, peptide, polypeptide, protein, carbohydrate, vaccine, adjuvant, lipid, or it may be a virus or cell, although it is not limited to any particular compound, biomolecule or entity. Such compositions have desirable controlled release properties such that an entrapped therapeutic agent or agents is released from the matrix under zero order, pseudo zero order or first order kinetics. The release characteristics are adjustable by selection of the appropriate phases, polymer(s), cross-linking agent(s), and excipients, among other factors.

The compositions of the invention may be prepared from a mixture of at least two phases, one of which is an aqueous phase and at least one of which comprises at least one therapeutic agent, and a polymer capable of being cross-linked, and forming cross-links between the polymer molecules to form a cross-linked matrix entrapping the at least one therapeutic agent. The cross-linking can be performed before, during, or after the matrix is administered to the animal. For example, the cross-linking reaction can be initiated in vitro, and the mixture, while undergoing cross-linking, may be injected into a bodily compartment of an animal, wherein the injected bolus continues to cross-link and harden in situ. In another embodiment, a cross-linked matrix after formation can be implanted or inserted into the location of the body from which delivery of the agent is desired. The compositions may also be introduced at either end of the gastrointestinal tract for transmucosal absorption.

The additional one or more phases other than the aqueous phase may be an oil (lipid) phase, or a solid phase. The oil (lipid) phase is preferably a compound or mixture thereof which is a liquid at the temperature at which the compositions of the invention are used, for example, for sustained release in the body or in an industrial setting. Non-limiting examples of suitable oil or lipid phase components include fatty acid esters, such as lower alcohol esters of myristic acid, high molecular weight fatty acids, and oils such as food oils, by way of illustration. The solid phase may be a compound or agent which is insoluble in the aqueous phase. It may also be a preformulated solid component, such as a microsphere or microfiber; in the case of microspheres, another phase, such as an aqueous or lipid phase, may be present within the solid microsphere.

The invention is also directed to a method for the controlled release of at least one therapeutic agent by administering to a site in the body a composition of the invention as described above.

The controlled release of the at least one therapeutic agent from the pharmaceutical composition of this aspect of the invention may occur as a consequence of diffusion from the at least one phase of the matrix wherein the active agent resides, or biodegradation of the matrix by an in-vivo degradation pathway such as via reducing agents, reductases, S-transferases, peptidases, proteases, non-enzymatic hydrolysis, esterases or thioesterases. As will be seen below, a remarkable and surprising finding herein is that the presence of multiple phases beneficially influences the controlled release characteristics of an active agent in the composition, whether or not the active agent is contained within any particular additional phase. The release may be zero order, pseudo-zero order or first order. Moreover, the ratio among the aforementioned types of stable and labile cross-linking bonds, among other factors, may be used to regulate the as persistence of the composition within the body and the release kinetics of the entrapped therapeutic agents. For example, a ratio of thioether, thioester and disulfide bonds may provide the proper release pharmacokinetics for a composition of the invention placed in a particular bodily site that is exposed to esterases as well as reducing activity.

The mixture may comprise one or more excipients that modulate one or more properties of the cross-linked matrix, such as swelling of the polymer, diffusion or partitioning of the therapeutic agent, or formation or maintenance of an emulsion. Such excipients include, by way of non-limiting example, mono- or divalent metal ions, anions or ionic polymers, proteins such as serum albumin, surfactants and polymers such as dextran. Moreover, components may be added to the composition to provide enhanced stability of any therapeutic agents contained therein, for example, proteinase inhibitors to maintain the stability of entrapped proteinaceous therapeutic agents. Such inhibitors may be present in the aqueous, lipid or solid phase, for example, in the form of microspheres. Slow release of the proteinase inhibitor from the microsphere protects the entrapped protein from attach by proteinases from the environment of the composition (such as one implanted in the body) from attaching the therapeutic agent.

The polymer of the materials and compositions of the invention comprises at least two functional or reactive groups which may particulate in cross-linking to form the matrix entrapping the agent, and may be a homopolymer or a copolymer. Any of a large number of such polymers or combinations may be used. The polymer may have a backbone such as but not limited to a polyalkylene oxide such as poly(ethylene glycol) (PEG or poly[ethylene oxide]), carboxymethylcellulose, dextran, modified dextran, polyvinyl alcohol, N-(2-hydroxypropyl)methacrylamide, polyvinyl pyrrolidone, poly-1,3-dioxolane, poly-1,3,6-trioxane, polypropylene oxide, a copolymer of ethylene/maleic anhydride, a polylactide/polyglycolide copolymer, a polyaminoacid, a copolymer of poly(ethylene glycol) and an amino acid, and a polypropylene oxide/ethylene oxide copolymer. Poly(ethylene glycol) is preferred. The foregoing polymer or polymers used to form the cross-linked matrix may independently have one or more types of functional groups which serve as sites for cross-linking. Such functional groups may be amino groups, carboxyl groups, thiol groups, and hydroxyl groups, by way of non-limiting examples. By way of example, the polymer may be derived from a poly(ethylene glycol) (PEG) derivative such as but not limited to α,ω-dihydroxy-PEG and α,ω-diamino-PEG, which may be cross-linked via hydroxy or amino groups. Another polymer with thiol functional groups may be prepared from, for example, α,ω-diamino-poly(ethylene glycol) and thiomalic acid; α,ω-dihydroxy-poly(ethylene glycol) and thiomalic acid; or α,ω-dicarboxy-PEG subunits and lysine, wherein free carboxy groups on the lysine residue are derivatized to provide thiol groups. In a particular embodiment, the poly(ethylene glycol) subunit size is from about 200 to about 20,000 Da. In a more preferred embodiment, the poly(ethylene glycol) subunit size is from about 600 to about 5,000 Da.

Preferably, the polymer comprises at least two thiol groups, and may be a homopolymer or a copolymer.

Such moieties may be cross-linked by reagents capable of forming covalent bonds between the functional groups, such as but not limited to homobifunctional and heterobifunctional cross-linking agents. A preferred moiety is a thiol group, and a preferred cross-linking agent is one that forms thioether bonds, such as a vinylsulfone or maleimide, but the invention is not so limiting. Other cross-linking reagents, such as a pyridyldithio-containing reagent, or oxidation, may be used to generate reducible cross-links. Combinations of cross-linking reagents may be used, as mentioned above, to provide a ratio of cross-link types which generate the desired as release characteristics of the composition. The preferred thiol-containing polymer may have from 2 to about 20 thiol groups. Preferably, the polymer has from about 3 to about 20 thiol groups, and most preferably, the polymer has from about 3 to about 8 thiol groups. In one embodiment, the thiol groups on the polymer are sterically hindered.

As noted above, the release rate of the therapeutic or other agent in the composition of the invention may be regulated by the biodegradability of the cross-linked polymer matrix. As multiply types of polymers and/or multiple types of cross-links may be formed, the degradation rate may be adjusted by varying the ratio or types of cross-links, and the stability or lability thereof, in the composition. For example, the ratio of reducing agent-sensitive disulfide bonds, esterase-sensitive ester bonds, and stable thioether bonds may be selected to provide the desired release kinetics of one or more entrapped agents.

As mentioned above, any of various conditions and/or reagents may be used to effect the cross-linking of the polymer, depending on the particular functional groups on the polymer. By way of non-limiting example, the conditions that cause cross-linking of the thiol groups on a thiol-containing polymer may be reaction in the presence of an oxidizing agent or reaction with a cross-linking agent. In the aspect of oxidation, the oxidizing agent may be by way of non-limiting example, molecular oxygen, hydrogen peroxide, dimethylsulfoxide, and molecular iodine. In the aspect where a cross-linking agent is used, the cross-linking agent may be a bifunctional disulfide-forming cross-linking agent or a bifunctional thioether-forming cross-linking agent. In a preferred embodiment, the cross-linking agent is a long-chain cross-linking agent, with a molecular weight of about 300 to about 5,000 Da. Non-limiting examples of suitable cross-linking agent include 1,4-di-[3′,2′-pyridyldithio(propionamido)butane]; α,ω-di-O-pyridyldisulfidyl-poly(ethylene glycol); a vinyl sulfone such as α,ω-divinylsulfone-poly(ethylene glycol); 1,11-bis-maleimidotetraethylene glycol; and α,ω-diiodoacetamide-poly(ethylene glycol).

For other functional groups or a combination of a thiol group and another group, any appropriate bifunctional cross-linking agent may be selected which will achieve the desired cross-linking of the functional groups and formation of the cross-linked polymer.

In another aspect, the polymer additionally comprises a functional group, which may derivatized for example with a label, such as a contrast/imaging agent, radionuclide, chromophore, fluorophore, red or near-infrared fluorophore, or nonradioactive isotope. In another embodiment, the label is a metabolically stable polymer component that after degradation of the polymer is detectable in the urine. In another embodiment, the cross-linking agent used to cross-link the polymer additionally comprises a functional group, such as a label.

In another related aspect, the delivering of at least one therapeutic agent to a bodily compartment under controlled release conditions is provided by situating in the bodily compartment a pharmaceutical composition comprising a matrix as described hereinabove. The bodily compartment may be subcutaneous, oral, intravenous, intraperitoneal, intradermal, subdermal, intratumor, intraocular, intravisceral, intraglandular, intravaginal, intrasinus, intraventricular, intrathecal, intramuscular, or intrarectal, by way of non-limiting examples. The composition of the invention may be provided to the bodily compartment by a route such as but not limited to subcutaneous, oral, intravenous, intraperitoneal, intradermal, subdermal, intratumor, intraocular, intravisceral, intraglandular, intravaginal, intrasinus, intraventricular, intrathecal, intramuscular, or intrarectal.

In yet a further aspect, the invention is directed to a method of preparing a cross-linked hydrogel drug depot, the method comprising: preparing a mixture comprising at least one therapeutic agent in a plurality of phases and a polymer system capable of forming a cross-linked hydrogel matrix, the polymer system comprising a first polymer having a plurality of functional groups, and a second polymer or long-chain compound having two or more functional or reactive groups; and forming linkages between the functional groups of the first polymer and the functional or reactive groups of the second polymer so as to form a cross-linked hydrogel matrix having a plurality of phases and the therapeutic agent physically entrapped therein. The plurality of phases are as described hereinabove. The first and second polymer may be the same or different. The first or second polymer may be a polyalkylene oxide, and either or both may be a homopolymer, a copolymer or a combination thereof. They may have one or more biodegradable linkages. In a preferred embodiment, one polymer comprises thiol groups and the other comprises vinylsulfone or maleimide groups. Reaction of the vinylsulfone or maleimide groups with the thiol groups forms cross-links. In another embodiment, the first and second polymers comprise thiol groups, and a homobifunctional thiol-reactive cross-linking agent is used to form cross-links. In these examples, the plurality of thiol groups may be between 2 and 20. The second polymer may be a long-chain cross-linking agent.

The releasing of a therapeutically effective amount of the therapeutic agent from the cross-linked hydrogel matrix may occur over a time course of three or more, five or more, ten or more, fifteen or more, or twenty or more days. Release of weeks to months by the compositions of the invention is also embraced herein. The controlled release profile may comprise a desired initial bolus release profile followed by a release profile such as but not limited to zero order, pseudo zero order, and first order.

The invention is further directed to a method of administering a therapeutic agent to a mammal, the method comprising: preparing a mixture comprising a hydrogel-forming polymer having two or more thiol groups, a cross-linker comprising two or more vinylsulfone or maleimide groups, and a therapeutic amount of drug and a plurality of phases; and injecting into a particular bodily compartment of the mammal with the mixture whereby a hydrogel drug depot is formed at the site of injection having said drug temporarily entrapped therein. Furthermore, the invention is also directed to a method of administering a therapeutic agent to a mammal by preparing a mixture comprising a hydrogel-forming polymer having two or more vinylsulfone groups, a cross-linker comprising two or more thiol groups, a therapeutic amount of drug, and a plurality of phases; and injecting said mammal with said mixture whereby a hydrogel drug depot is formed at the site of injection having the drug temporarily entrapped therein. In either of the foregoing methods, the cross-linker may comprise a hydrogel forming polymer, and may farther comprise releasing a therapeutically effective amount of the therapeutic agent from said hydrogel drug depot over a time course of three or more days. The injecting may be subcutaneous.

In a further embodiment, the present invention is directed to a hydrogel drug depot comprising a therapeutic agent physically entrapped within a polymer matrix comprising a thioether cross-linked hydrogel matrix and a plurality of phases. The hydrogel matrix may comprise a polyalkylene oxide, which may be a homopolymer, copolymer or combination thereof of poly(ethylene glycol) or derivative thereof. The polymer matrix may comprise a controlled release kinetic profile characterized by release of a therapeutically effective amount of the therapeutic agent from the thioether cross-linked hydrogel matrix over a time course of three or more, five or more, ten or more, fifteen or more, or twenty or more days. The controlled release kinetic profile may comprise an initial bolus release profile followed by a release profile such as zero order, pseudo zero order, or first order. The hydrogel depot may comprise one or more excipients that modulate one or more properties of the thioether cross-linked hydrogel matrix, such as diffusion, swelling, partitioning of the therapeutic agent, or formation or maintenance of an emulsion. Such excipients include, by way of non-limiting example, mono- or divalent metal ions, anions or ionic polymers, proteins such as serum albumin, surfactants, and polymers such as dextran. A proteinase inhibitor may be used. One or more may be present in the composition.

The therapeutic agent of the hydrogel drug depot may be, by way of non-limiting example, a small organic molecule, nucleic acid, peptide, polypeptide, protein, carbohydrate, vaccine, adjuvant, or lipid.

The cross-linked hydrogel matrix of the hydrogel drug depot may be formed by cross-linking a first polymer containing two or more thiol groups with a second polymer or long-chain compound containing two or more vinyl sulfone groups. The first polymer may comprise a molecular weight of 200 to 20,000 Daltons; the second polymer or long-chain compound may comprise a molecular weight of 100 to 5,000 Daltons. The first polymer may comprise between 2 and 20 thiol groups. The first and second polymers may be in a defined molar ratio for controlling the controlled release kinetic profile of the hydrogel drug depot. The thioether cross-linked hydrogel matrix may comprise one or more biodegradable linkages.

In yet a further aspect, the invention is directed to a hydrogel drug depot system comprising a compound of interest, a plurality of phases, a first polyalkylene oxide polymer containing two or more thiol groups, a second polyalkylene oxide polymer containing two or more vinyl sulfone groups that are capable of covalently bonding to one another to form a thioether cross-linked hydrogel matrix, the hydrogel drug depot system having a controlled release kinetic profile characterized by sustained release of the compound of interest from the thioether cross-linked hydrogel matrix over a time course of three or more days and in some embodiments extending up to several months. The polyalkylene oxide may be poly(ethylene glycol) or a derivative thereof, the hydrogel matrix may comprise one or more biodegradable linkages, such as but not limited to an ester linkage. The hydrogel drug depot may have a controlled release kinetic profile comprising an initial bolus release profile followed by a release profile such as zero order, pseudo zero order, or first order.

The invention is also directed to a kit for forming a hydrogel drug depot comprising an agent of interest such as a therapeutic agent or a diagnostic agent, the kit including an aqueous phase, an oil or lipid phase, a surfactant, a polymer with two or more functional groups, and a cross-linking agent capable of forming a cross-links among the functional groups. In the use of the kit, a therapeutic agent is added to the components and cross-linking induced, in accordance with one of the aforementioned processes of forming the matrix in vitro, or forming it in situ by injecting the components soon after mixing, such that the matrix is not yet polymerized and can pass through a needle or cannula, and full cross-linking occurs in situ.

In a particular embodiment, the invention is directed to a kit for forming a hydrogel drug depot comprising an agent of interest such as a therapeutic agent or a diagnostic agent, a polymer having two or more thiol groups, and a low molecular weight, polymer or long-chain cross-linking compound having two or more vinylsulfone groups, and a plurality of phases, wherein said polymer and said cross-linker are capable of covalently bonding to one another under physiological conditions to form a thioether cross-linked hydrogel matrix so as to entrap the agent of interest therein. The hydrogel matrix may comprise polyalkylene oxide. In the foregoing kit, the polyalkylene oxide may be a homopolymer, copolymer or combination thereof of poly(ethylene glycol) or derivative thereof. The polymer matrix comprises a controlled release kinetic profile characterized by release of a therapeutically effective amount of the therapeutic agent from the thioether cross-linked hydrogel matrix over a time course of three or more, five or more, ten or more, fifteen or more, or twenty or more days. Release over weeks to months is also embodied herein. The controlled release kinetic profile may comprise an initial bolus release profile followed by a release profile such as zero order, pseudo zero order, or first order. The kit may include one or more excipients that modulate one or more properties of the thioether cross-linked hydrogel matrix, such as, but not limited to diffusion and swelling. The therapeutic agent is selected from the group consisting of small organic molecule, nucleic acid, peptide, polypeptide, protein, carbohydrate, vaccine, adjuvant, and lipid. The diagnostic agent may be a contrast/imaging agent, radionuclide, chromophore, fluorophore, red or near-infrared fluorophore, or a non-radioactive isotope. The kit may also have other types of polymers and cross-linkers.

In the aforementioned kits, the polymer may have a molecular weight of 200 to 20,000 Daltons. The cross-linking agent, whether a small molecule, polymer or long-chain compound, may have a molecular weight of 100 to 5,000 Daltons. The polymer may have between 2 and 20 thiol groups. The polymer and cross-linking agent may be provided in preformed aliquots for admixing to generate a defined molar ratio of the first and second polymers for controlling the controlled release kinetic profile of the hydrogel drug depot.

In another aspect of the invention, a method of producing a kit according to the above may be performed by assembling in the kit an agent of interest such as a therapeutic agent or a diagnostic agent, a first polymer having two or more thiol groups, and a second polymer or long-chain compound having two or more vinyl sulfone groups, wherein the first polymer and the second polymer or long-chain compound are capable of covalently bonding to one another under physiological conditions to form a thioether cross-linked hydrogel matrix so as to entrap the agent of interest therein.

These and other aspects of the present invention will be better appreciated by reference to the following drawing and Detailed Description.

### BRIEF DESCRIPTION OF THE DRAWING

FIG. 1 shows the in vitro release of quinine sulfate monohydrate from two different formulations of the thiol containing polymer hydrogel, formulation I having an aqueous phase, and oil phase and a solid phase, and formulation II having an aqueous phase and a solid phase.

FIG. 2 shows another in vitro release of quinine sulfate monohydrate from two different formulations of the thiol containing polymer hydrogel.

FIG. 3 shows the in-vivo release of quinine sulfate monohydrate from two different formulations of the thiol containing polymer hydrogel, the Rgel formulation having a aqueous phase and a solid phase, and the Egel formulation an aqueous, an oil phase and a solid phase. The insert shows the initial release profiles of the two formulations.

FIG. 4 shows the in-vitro release of salmon calcitonin from two different formulations of the thiol containing polymer hydrogel of the invention.

FIG. 5 shows the in-vivo release of salmon calcitonin from two different formulations of the thiol containing polymer hydrogel, Rgel formulation having a single aqueous phase and Egel formulation an aqueous and an oil phase.

### DETAILED DESCRIPTION OF THE INVENTION

The present invention provides new and useful materials including compositions and pharmaceutical compositions, and methods for their preparation and administration, based upon the surprising and remarkable discovery by the inventors herein that cross-linking of a polymer and formation of a cross-linked polymer matrix in a multiple phase system comprising an aqueous phase and at least one other phase, preferably at least an oil (lipid) phase, provides a material with various heretofore unknown properties useful for a variety of industrial and medical applications, among others. By physically entrapping, for example, an active agent in the composition in one or more of the phases, particular storage and handling features of the material may be provided, and such materials may be prepared with desirable release properties for the one or more entrapped agents. As will be elaborated upon below, such multiple-phase systems include an aqueous phase and a solid phase, or an aqueous phase and an oil (lipid) phase, or an aqueous, oil (lipid) and solid phase. Furthermore, the aqueous and oil (lipid) phases may be provided in the form of an emulsion. An emulsion is a preferred multiple phase system. The terms “solution,” “mixture,” and “suspension” are used interchangeably to refer to the compositions herein comprising a plurality of phases before the matrix is cross-linked, such as a suspension of solid particles in an aqueous phase or an oil-aqueous phase emulsion. Oil and lipid are interchangeably used to refer to a liquid, water-insoluble phase. Various agents, other than the active agent(s), herein termed excipients, may be included in the compositions to enhance the formation or stability of the emulsion, to maintain the separate phases, or to modulate the partitioning of an active agent among the phases and to modulate the storage (retention) or release characteristics of the composition. Moreover, multiple oil phases or solid phases may be present in the aqueous phase, for example, two or more types of oils or solid particles of different compositions. In a preferred embodiment, an emulsion is formed from an aqueous and an oil phase, with a surfactant or detergent.

Certain of the compositions or matrices of the invention may also be referred to as hydrogel compositions or matrices as they comprise a hydrophilic polymer in an aqueous phase, and exhibit a gel or semisolid consistency.

By way of example, an active agent as described above may be any agent desirably prepared in a composition with the properties described hereinabove, such as an industrial or household chemical or reagent, for example, a perfume, flavoring agent, sweetener, antiseptic, antifouling agent, pesticide, etc., for which storage, transport, or preferably controlled release from a composition of the invention is desired. A pharmacologically-active agent is preferred, such as is used for the prophylaxis or therapy of a disease or condition, and wherein the composition or matrix of the invention is ingested or implanted in an animal body for the delivery of the therapeutic agent(s) entrapped therein. As will be seen below, in one embodiment, the cross-linked matrix of the invention is formed in situ by injection of the components before or during formation of the cross-linked polymer. Moreover, the invention is not so limiting as to the nature of the agent physically entrapped in the compositions herein.

By way of the non-limiting example of a pharmaceutically-useful agent (therapeutic agent) in the instant composition, the composition may be prepared to release the agent(s) with a controlled release kinetic profile in vivo, such as zero order, pseudo zero order or first order release. A preferred release is a constant rate of release over time. As will be seen below, the compositions are prepared using a polymer with functional and/or reactive groups that may participate in cross-linking reactions, and another compound, such as but not limited to a polymer, which reacts with and cross-links the polymer with functional or reactive groups. A mixture of the multiple phases, polymer, and optional active agent(s) and excipient(s) is prepared, and then cross-linking of the polymer is initiated by placing the mixture under conditions which cause cross-linking, such as exposure to a cross-linking agent, heating, cooling, polymerization-inducing radiation, etc. Moreover, the compositions may be prepared such that the material may be readily deposited in a bodily compartment without the need for surgery, by injecting through a needle or cannula the composition of the invention while in liquid form, the cross-linking reaction solidifying the composition in situ.

The compositions of the invention comprise a polymer matrix prepared from polymers bearing moieties, such as thiol moieties, which are capable of being cross-linked by any of a number of processes, such as oxidation or by use of a bifunctional cross-linking agent, to physically entrap the therapeutic agent within the cross-linked polymer. The matrices are prepared by cross-linking the polymer in the presence of the therapeutic agent(s), such that entrapment occurs during cross-linking. The invention is directed to compositions and pharmaceutical compositions prepared by these methods, methods of preparing the compositions by cross-linking the polymer to entrap the agent therein, as well as to methods for administering the composition to an animal, for instance, by injecting the composition of the invention into an animal during the process of cross-linking such that the mixture is liquid or semi-solid during injection, but soon after injection completes the cross-linking process and forms the matrix (depot) with the aforementioned release characteristics. Thus, the cross-linking of the polymer may be performed during the manufacture of the composition, which is subsequently administered to or implanted at the desired site; in another embodiment, a mixture of the to; therapeutic agent(s), the polymer and the cross-linking agent is administered to the desired site at the time of or just after initiation of the cross-linking reaction, such that the mixture can be readily deposited at the desired site, and the cross-linking subsequently occurs or is completed in the bodily compartment to form the matrix. In all of the foregoing examples, the agent or agents and multiple phases are physically entrapped within the cross-linked polymer.

The term agent or active agent refers to the substance for which the matrix of the invention may be used to hold, deliver, stabilize, release, carry, transport, store, or otherwise handle for any purpose for which the agent may be used. As noted herein, a preferred agent is a therapeutic agent, but the agent may be any agent. The term therapeutic agent should not be considered limiting to medically useful agents. The composition of the invention may not comprise any active agent, the cross-linked multiphase composition having useful properties itself.

As used herein, a phase refers to a distinct liquid or solid phase, such as an aqueous, solid, or oil phase, and as will be seen below, a composition or matrix of the invention comprises two or more phases. One of the phase is an aqueous phase. The agent may be present in one or more phases. For example, a matrix comprising an poorly water-soluble agent may comprise a solid phase (the agent), and an aqueous phase. A matrix may comprise an emulsion of an aqueous phase and an oil phase, the agent present in the aqueous phase, the oil phase, or both phases. A matrix comprising three phases may comprise an emulsion with a solid phase, the solid phase present in the aqueous phase, the oil phase, or in both. Moreover, multiple agents may be present in the compositions of the invention; for example, multiple agents suspended in an aqueous phase; multiple oil-soluble agents present in single or multiple oil phases, such as by the mixture of two emulsions, each prepared from a different oil-soluble agent, before cross-linking of the polymer. As noted above, an excipient may be used to enhance or assist in the formation of the multiple phase system, for example, by use of a surfactant such as a detergent to form the emulsion; the use of a monovalent or polyvalent metal ion or polymer to aid in the insolubilization of the active agent, or an excipient to alter the pH or other properties to partition the active agent in one or more phases and regulate or modulate its release from the one or more phases to the desired outside environment in which the composition of the invention resides. It may also include a proteinase inhibitor which prevents degradation of a proteinaceous agent within the matrix. The term excipient is used herein to refer to any compound, substance, agent, material, etc., which is not the active agent whose release is provided by the instant compositions, but agents which regulate or modulate the release, including formation of the emulsion or the matrix in general. The desired release may be no release. The excipient may be retained in the composition during the erosion or diffusion of the active agent from the composition or it may be co-released along with the active agent including in a complex with the agent, but permits the active agent to have its desired activity or function after release from the instant composition.

The phases present in the cross-linked matrix include an aqueous phase and at least one additional phase. In a preferred embodiment, the additional phase is an oil phase, such as ethyl myristate as described in the examples. Other choices of oils for the oil phase are one or more compounds which are liquid at the temperature at which the compositions of the invention are used, for example, for sustained release in the body or in an industrial setting. Non-limiting examples of suitable oil or lipid phase components include fatty acid esters, such as lower alcohol esters of caproic acid (C6), caprylic acid (C8), capric acid (C10), undecanoic acid (C11), lauric acid (C12), tridecanoic acid (C13), myristic acid (C14), and palmitic acid (C16). Non-limiting examples of such esters include but is not limited to caproic acid ethyl ester, caprylic acid ethyl ester, capric acid ethyl ester, undecanoic acid ethyl ester, lauric acid ethyl ester, tridecanoic acid ethyl ester, myristic acid ethyl ester, and palmitic acid ethyl ester. Other choices for the oil phase include triglycerides that are liquid at room temperature, such as triacetin (C2), tributyrin (C4), tricaproin (C6), and tricaprylin (C8). Also, fatty alcohols which are liquid at room temp may be used, such as 1-octanol (C8) and 1-decanol (C10). Other examples include unsaturated fatty acids such as cis-11,14-eicosadienoic acid, and unsaturated fatty acid esters such as cis-11,14-eicosadienoic acid ethyl ester. Food oils such as the vegetable oils corn oil, olive oil, safflower oil, and canola oil may be used. There are merely illustrative of various water-immiscible liquids that may be used as an oil phase of the compositions of the invention, and that may be prepared as an emulsion in combination with an aqueous phase in one embodiment of the invention.

Inclusion of a surfactant such as sodium dodecyl sulfate (SDS) in the aqueous phase provides for the rapid formation of an emulsion of the aqueous and oil phases. Multiple different oil phases may be present. In another embodiment, a solid phase, such as a water-insoluble therapeutic agent, may be present in the aqueous phase or in the emulsion. The oil phased may be any water-immiscible liquid. Low molecular weight alcohol esters of fatty acids are preferred oil components, but the invention is not so limited.

In another embodiment, compositions are described which comprise a cross-linked polymer matrix entrapping at least one therapeutic agent, the matrix comprising a plurality of phases, at least one being an aqueous phase. Methods for preparing the latter compositions are also described. Thus, in this aspect, solid particles of a poorly water soluble therapeutic agent may be suspended in an aqueous phase, the foregoing entrapped within the matrix. Continuous release from the matrix of the molecules of the therapeutic agent that have become dissolved in the aqueous phase will result in continuous solubilization of the suspended therapeutic agent into the aqueous phase, thus replenishing the therapeutic agent in the aqueous phase. In a similar manner, a bi-phasic system comprising an oil-water emulsion wherein the therapeutic agent is present in the oil phase or aqueous phase, either dissolved or suspended (based upon its solubility), also represents a controlled release system in which, for example, an oil-soluble therapeutic agent with limited water solubility is entrapped in the matrix; release of the agent from the aqueous phase permits redistribution of the agent from the oil phase into the aqueous phase. As will be noted in more detail below, various excipients may be included in the compositions herein to aid in the formation and/or stability of the composition with multiple phases, particularly emulsions, as well as regulate the partitioning of the agent among the phases, which further modulate the release characteristics and kinetics of the compositions.

The polymer which is cross-linked to entrap the therapeutic agents may be any cross-linkable polymer, which bears two or more functional or reactive groups capable of participating in a cross-linking reaction to form a matrix of the invention. Such functional groups include but are not limited to amino, carboxyl, thiol and hydroxyl groups, or combinations thereof; reactive groups include vinylsulfone, maleimide, pyridyldithio, and other moieties capable of reacting with the aforementioned functional groups, among others. A preferred polymer is one on which at least two thiol groups are present and is cross-linked with a thiol-reactive bifunctional cross-linking reagent in the presence of the therapeutic agent, thus forming a cross-linked polymer with the therapeutic agent physically entrapped therein. Selection of the appropriate polymer, the concentration in the matrix, the extent of functional groups capable of participating in cross-linking, the type of cross-linking agent, and the extent of cross-linking, and other factors will be governed by such factors as the amount of therapeutic agent present in the composition, the number of phases and the relative amounts of the phases including the phase in which the polymer is present, in order to achieve the desired controlled release properties of the composition, or retention of the active agent within the composition. In a preferred embodiment for pharmaceutical agents, and in particular for proteins, the cross-linking is accomplished by using sulfur chemistry for cross-linking the polymer, thereby avoiding reaction of virtually all amino acid and carbohydrate side chains of, for example, a protein therapeutic agent undergoing entrapment in the matrix. Although sulfur chemistry is the basis of the cross-linking preferably used in this invention, disulfide bonds already present in a particular protein would be non-reactive under the cross-linking conditions. Also, the sulfur atom in the thioether side chain of methionine residues in the protein drug would be nonreactive. Delivery of small-molecule drugs, peptides, proteins, polysaccharides, and polynucleotides including antisense nucleotides are achievable using the methods described herein. Proteins containing free thiol groups (cysteine residues that are not in disulfide linkage), might not be suitable for use in their native form in this invention, and may need to be derivatized or otherwise protected during the entrapment process. Similar considerations are given to other non-protein therapeutic agents which are used in the present invention.

One advantage to using sulfur chemistry in general, and reducible cross-links in particular as may be produced from oxidation of the thiol groups on the polymer or by use of a reducible bond-forming cross-linking agent such as one containing a pyridyldithio (pyridyldisulfidyl) group is that a cross-linked matrix formed in situ in a bodily compartment or other relatively inaccessible area may be readily and facilely removed by exposing the cross-linked composition in situ to a reducing agent, whereupon the cross-links are broken and the composition can be flushed or extracted from the site. This may be achieved, for example, when an implanted release composition has achieved its desired goal of controlled releasing a therapeutic agent over time, or for early removal of a device. Of course, since any remaining therapeutic agent entrapped within an implanted device will be subject to rapid release when the cross-linked polymer is rapidly depolymerized, considerations must be given to remove the device from the site to avoid an unwanted bolus release.

However, the invention is not so limiting to sulfur chemistry to form the cross-linked matrix, and polymers and cross-linking agents which achieve the desired properties may be achieved using other functional and reactive groups, including both polymeric and non-polymeric cross-linking agents.

With regard to pharmaceutically-useful active agents, for long term therapy (days, weeks or months) and/or to maintain the highest possible drug concentration at a particular location in the body, the present invention provides a sustained release depot formulation with the following preferred but non-limiting characteristics: (1) the process used to prepare the matrix does not chemically or physically damage the therapeutic agent, in particular proteins, thereby avoiding protein inactivation or rendering the protein immunogenic; (2) the matrix maintains the stability of a therapeutic agent against denaturation or other metabolic conversion by protection within the matrix until release, which is important for very long sustained release; (3) the entrapped therapeutic agent is released from the depot at a substantially uniform rate, following a kinetic profile, and furthermore, a particular therapeutic agent can be prepared with two or more kinetic profiles, for example, to provide a loading dose and then a sustained release dose; (4) the desired release profile can be selected by varying the components and the process by which the matrix is prepared; and (5) the matrix is nontoxic and degradable.

In the preferred but non-limiting embodiment, the cross-linked matrix of the present invention entrapping at least one therapeutic agent is prepared by cross-linking a polymer for example on which at least two thiol groups are present, by any one of various means, in the presence of the therapeutic agent to be physically entrapped. Various polymers on which at least two thiol groups are present are suitable for the use herein. The polymer on which at least two thiol groups are present may be prepared, for example, by the reaction or derivatization of a particular polymer that does not contain thiol groups, with a thiol-containing compound, or a compound to which thiol moieties may be attached. A polymer may be prepared which has reactive terminal ends or functional groups on the ends of the polymer chain which may be subsequently derivatized to attach thiol groups. A copolymer may be prepared with repeating or alternately repeating thiol groups or functional groups which may be subsequently derivatized to have thiol groups. The extent of derivatization to provide thiol groups may be tailored to the requirements of the matrix to be formed. The foregoing examples of the types of suitable polymers is not intended to be limiting, but to be illustrative of the varieties of polymers and polymer derivatives that may be used in the practice of the invention.

In the case of thiol groups, to participate in cross-linking, the polymer has at least two thiol groups to participate in the formation of cross-links. For example, the polymer on which at least two thiol groups are present may have from 2 to about 20 thiol moieties. In a preferred embodiment, the polymer has from 3 to about 20 thiol moieties, and in a most preferred embodiment, the thiol containing polymer has from 3 to about 8 thiol moieties. These numbers of functional groups on the polymer are equally applicable to other selections of functional groups, such as amino, carboxyl and hydroxy groups, by way of non-limiting examples.

Examples of suitable subunit polymers for the preparation of the polymer on which at least two thiol groups are present include both homopolymers or copolymers. By way of non-limiting example, suitable polymers, which may be chemically modified to comprise thiol groups, include polyalkylene oxides such as poly(ethylene glycol) [also known as polyethylene glycol or PEG, polyethylene oxide or PEO], carboxymethylcellulose, dextran, polyvinyl alcohol, N-(2-hydroxypropyl)methacrylamide, polyvinyl pyrrolidone, poly-1,3-dioxolane, poly-1,3,6-trioxane, polypropylene oxide, a copolymer of ethylene/maleic anhydride, a polylactide/polyglycolide copolymer, a polyaminoacid, a copolymer of poly(ethylene glycol) and an amino acid, or a polypropylene oxide/ethylene oxide copolymer. Such polymers are then derivatized or further polymerized to introduce thiol groups; chemical modification of the polymer may be necessary as a step prior to the further derivatization to incorporate thiol groups. For example, a polymer of the present invention may be derived from a poly(ethylene glycol) (PEG) derivative, for example, α,ω-dihydroxy-PEG or α,ω-diamino-PEG, but other derivatives are embraced herein. The polymer comprising thiol groups may be, for example, a polymer of α,ω-diamino-poly(ethylene glycol) and thiomalic acid; a polymer of α,ω-dihydroxy-poly(ethylene glycol) and thiomalic acid; or a polymer of α,ω-dicarboxy-PEG subunits and lysine wherein the free carboxy groups on the lysine residues are derivatized to form thiol groups. These polymers are only examples of possible choices, as the skilled artisan will be aware of numerous alternatives. As will be noted below, the selection of the polymer, or combinations thereof, will be guided by the desired properties of the final product, particularly the duration of release of the therapeutic agent and the release kinetics. As will also be noted below, a product of the invention may comprise more than one polymer component in order to provide two or more different release characteristics. Of course, more than one therapeutic agent may be included.

In one particular embodiment, a polymer of the present invention is derived from a poly(ethylene glycol) (PEG) derivative, for example, α,ω-dihydroxy-PEG or α,ω-diamino-PEG, but other derivatives are embraced herein. Examples of such polymers with particular molecular weights include α,ω-dihydroxy-PEG_{3,400}; α,ω-dihydroxy-PEG_{1,000}; α,ω-diamino-PEG_{3,400}; and α,ω-diamino-PEG_{1,000}. PEG is known to be a particularly nontoxic polymer. These derivatized PEG subunit polymers may be used as amino- and hydroxy-containing polymers for cross-linking, or may be further derivatized, for example, to prepare the polymer on which at least two thiol groups are present by derivatization with thiomalic acid. Thiomalic acid (also known as mercaptosuccinic acid) may be replaced by dimercaptosuccinic acid, thereby doubling the number of sites available for cross-linking. Increasing the extent of cross-linking the matrix results in a gel with smaller pores.

As will be shown in an example below, to prepare the polymer on which at least two thiol groups are present from these reactants, the thiol group of thiomalic acid is first protected by reaction with trityl chloride, to produce trityl-thiomalic acid. Subsequently, the polymer on which at least two thiol groups are present is prepared from the trityl-thiomalic acid and, for example, α,ω-dihydroxy-PEG. Under suitable conditions, a carbodiimide is used to condense the α,ω-dihydroxy-PEG with the protected thiomalic acid. After condensation, the trityl group is removed by treatment with trifluoroacetic acid (TFA).

In another example, a polymer of α,ω-dicarboxy-PEG and lysine may be prepared, and subsequently the free carboxy groups on the lysine residues are derivatized to form thiol groups. These examples are provided by way of illustration only and such methods for adding thiol groups to a polymer are known to those skilled in the art.

In a preferred embodiment using PEG as the subunit for preparing the polymer on which at least two thiol groups are present, the poly(ethylene glycol) subunit size for the polymer may be from about 200 to about 20,000 Da; preferably, the subunit size is from about 600 to about 5,000 Da. As mentioned above, the polymer of the present invention has from 2 to about 20 thiol groups; preferably from about 3 to about 20 thiol groups, and most preferably, from about 3 to about 8 thiol groups.

The thiol groups on the polymer on which at least two thiol groups are present may be sterically hindered. It has been found that a polymer on which at least two thiol groups are present with sterically hindered thiol groups tends to be nonreactive with disulfide bonds in the therapeutic agent, particularly a protein, and thus does not interfere with the intramolecular disulfide bonds in the protein. Furthermore, steric hindrance governs the rate at which reductive cleavage of the polymer occurs in vivo. Thus, for the entrapment of proteins or other therapeutic agents with disulfide bonds, a polymer on which at least two thiol groups are present, sterically hindered thiol groups may be preferred. Such sterically hindered thiol groups are also preferred when increased resistance to reductive cleavage is desired, for example in a longer controlled release formulation. Based on the knowledge of the therapeutic agent and the particular controlled release characteristics desired at the site of administration of the matrix, the skilled artisan will be able to design a matrix with the desired characteristics. Examples of such sterically hindered thiol groups include thiomalate, as used in the above example.

A matrix of the present invention may be prepared by cross-linking the polymer on which at least two thiol groups are present in the presence of the therapeutic agent. The cross-linking of the polymer on which at least two thiol groups are present may include disulfide bonds, thioether bonds, and combinations thereof. Other means of covalent bond formation of thiol groups in the thiol-containing polymer to effect cross-linking will be known to the skilled artisan and are considered within the scope and spirit of this invention.

In one example, reaction of the polymer on which at least two thiol groups are present in the presence of an oxidizing agent forms disulfide cross-links. This may be achieved by molecular oxygen, hydrogen peroxide, dimethyl sulfoxide (DMSO), or molecular iodine. In other embodiments, the cross-linking may be carried out by reaction with a bifunctional disulfide-forming cross-linking agent, or reaction with a bifunctional thioether-forming cross-linking agent. Such cross-linking agents may have a molecular weight of about 300 to about 5,000 Da, and may be a polymeric cross-linking agent.

For example, the PEG-thiomalate polymer described above may be cross-linked with the non-polymeric cross-linking agent 1,4-di-[3′,2′-pyridyldithio(propionamido)-butane]. Alternatively, a polymeric cross-linking agent such as α,ω-di-O-pyridyldisulfidyl-poly(ethylene glycol); α,ω-divinylsulfone-poly(ethylene glycol); or α,ω-diiodoacetamide-poly(ethylene glycol) may be used. Another thioether-forming thiol group crosslinker is 1,11-bis-maleimidotetraethylene glycol, abbreviated BM(EG)₄ or BM[PEO]₄, available from Pierce. Examples of the cross-linking reaction are provided in the examples below; the skilled artisan will be aware of numerous other agents capable of forming the suitable matrix. As noted above, the selection of the cross-linking agent is guided by the desired characteristics of the matrix product, i.e., the controlled release kinetic profile and the duration of release. These factors, as well as the potential reactivity of the cross-linking agent with reactive moieties on the therapeutic agent, must be taken into consideration in selecting the appropriate polymer, and cross-linking agent in the preparation of the product. And as mentioned above, the presence of reducible cross-links, such as derived using a pyridyldithio cross-linker or oxidation, may be useful in whole or in part for regulating the release characteristics of the composition, or for depolymerizing the composition for removal after use.

The therapeutic agent physically entrapped in the matrix of the present invention is a compound capable of being entrapped and then released in a controlled manner from the matrix. A wide variety of both high molecular weight and low molecular weight compounds are suitable, and as will be noted below, a compound not suitable because of its small size may be made suitable by appropriate modification by for example, polymerization or conjugation to a polymer. The therapeutic agent may be a small-molecule drug, protein, peptide, polysaccharide, polynucleotide, or any other compound that may be entrapped in the matrix of the present invention and subjected to controlled delivery in vivo. It is noted that a further advantage of the present invention is that the matrix protects the therapeutic agent from degradation or other metabolic processing. The agents may be for the prophylaxis or treatment of a condition or disease, or for the purpose of providing controlled delivery of any suitable agent.

For example, when polymers of the following PEG polymers are prepared with thiomalic acid, and then similarly cross-linked, certain properties of the polymer are obtained. The α,ω-dihydroxy-PEG_{3,400} polymer subunit is conjugated via an ester bond to the thiomalic acid, and the resulting product is loosely cross-linked. Likewise, a loosely cross-linked product is formed from thiomalic acid and α,ω-diamino-PEG_{3,400}, the thiomalic acid linked through an amide bond to the PEG subunit. In contrast, α,ω-dihydroxy-PEG_{1,000} linked to thiomalic acid through an ester bond is tightly cross-linking, as is α,ω-diamino-PEG_{1,000}, through an amide bond.

The agents may be industrial chemicals or compounds, household chemicals such as cleaners, perfumes or other odorants, deodorants, fertilizers or plant food, foodstuffs such as slow-release food for aquarium fish, therapeutic agents, etc. In a preferred embodiment, the agent is a therapeutically or prophylactically effective agent, generally referred to herein as a therapeutic agent, for controlled release in a bodily compartment of an animal, such as a mammal, preferably a human.

The therapeutic agents of the invention are not limited to any particular structural type or therapeutic class, and may include small-molecule drugs, peptides and proteins, carbohydrates, and nucleic acids, to name some non-limiting structural compound classes. Small molecule drugs may include, for example, anticancer drugs, cardiovascular drugs, antibiotics, antifungals, antiviral drugs, AIDS drugs such as HIV-1 protease inhibitors and reverse transcriptase inhibitors, antinociceptive (pain) drugs, hormones, vitamins, anti-inflammatory drugs, angiogenesis drugs, and anti-angiogenesis drugs. Among the examples of suitable therapeutic agents are proteins. This includes proteins, peptides, modified proteins and peptides, and conjugates between proteins or peptides and other macromolecules. The protein may be a recombinant protein. For example, candidate agents include erythropoietin, α-interferon, growth hormone and antibodies. Erythropoietin is administered over long periods to promote the formation of red blood cells, such as in conditions including renal failure or cancer therapy-induced anemia, α-Interferon is used to treat certain viral diseases (e.g. hepatitis) and cancers (e.g. hairy cell leukemia). Growth hormone is used for pituitary dwarfism. These compounds are therapeutically effective for certain indications when administered at low doses over an extended period of time, making them good candidates for controlled delivery from a depot administration as described herein, as they otherwise are administered by injection.

Another group of suitable protein agents are antibodies and antibody fragments, such as those directed against tumor-specific antigens and against inflammatory response proteins such as tumor necrosis factor and interleukin 1, are additional examples of proteins that may be used in the practice of the present invention. As such products usually require frequent parenteral administration, such as by injection, a matrix with an antibody delivered by controlled release provides convenience. The antibody is protected from biodegradative machinery while in the matrix.

Another example of a class of therapeutic agents are polysaccharides. Examples include sulfated polysaccharides, such as heparin or calcium spirulan. Heparin is an anticoagulant for which long-term therapy is indicated in various hypercoagulation disorders and for prophylactic use. Chronic anticoagulation therapy is indicated, for example, postoperatively to prevent stroke and pulmonary embolism, and in deep vein thrombosis. Calcium spirulan is a potent antiviral agent against both HIV-1 and HSV-1 (herpes simplex virus) (Hayashi et al., 1996, AIDS Research & Human Retroviruses. 12(15):1463-71).

A further example of suitable therapeutic agents is polynucleotides, such as antisense oligonucleotides. These may be delivered to a particular site within the body using the methods described herein, for sustained delivery to target cells or tissues. Such polynucleotides may be in the form of vectors, gene therapy agents or antisense oligonucleotides. These may be delivered to a particular site within the body using the methods described herein, for sustained delivery to target cells or tissues. Such gene therapy agents include but are not limited to a gene encoding a particular protein or polypeptide domain fragment either as a naked plasmid or introduced in a viral vector. Such vectors include, for example, an attenuated or defective DNA virus, such as but not limited to herpes simplex virus (HSV), papillomavirus, Epstein Barr virus (EBV), adenovirus, adeno-associated virus (AAV), and the like, including retroviral vectors. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus, a particular tissue can be specifically targeted.

Another example of a therapeutic agent embraced by the invention herein is a vaccine. Administration to an animal of an immunogen in the matrix of the present invention with the proper controlled release kinetics provides the immune system with an antigen for the development of a humoral and/or cellular response. Indeed, fluid flow carrying the released antigen from a subcutaneous depot of the present invention is through lymphatic tissue where the immune response to that antigen may occur.

The foregoing lists and descriptions of therapeutic agents are merely illustrative of examples of various therapeutic agents which may be present singly or in combination in the pharmaceutical compositions of the invention.

It will be noted that the judicious placement of the matrix of the present invention will permit targeted delivery to a particular site within the body, and furthermore, allow a higher concentration of the therapeutic agent to contact a particular site than achievable if the same therapeutic agent is administered systemically. In particular, administration of an agent which induces apoptosis in dysproliferative conditions, such as a tumor, may be performed by the placement (herein termed administration) of the matrix in the proximity of the tumor, thus delivering the therapeutic agent proximal to the tumor. The same strategy is used for proximal delivery of therapeutic agents to other particular body sites or compartments, such as through the skull into the brain.

In another embodiment of the present invention, the therapeutic agent may be derivatized to increase its molecular weight, such that it may be better entrapped by and released from the matrix. The derivatization may be, by way of non-limiting example, polymerization or conjugation to poly(ethylene glycol). Such methods of conjugation or polymerization are known to the skilled artisan.

Alternatively, as described above, the therapeutic agent may be prepared as a suspension of a solid in an aqueous solution of the matrix-forming polymer, thereby becoming entrapped during cross-linking within the matrix in the form of solid particles. Being considerably larger than individual molecules, these solid particles of therapeutic agent will be securely entrapped due to the relatively small pore size of the gels. A given size distribution of the solid particles may be attained by methods known to those skilled in the art. In another embodiment, a carrier molecule, such as human serum albumin (HSA), may be admixed with the therapeutic agent, such as by lyophilizing a solution containing HSA and said therapeutic agent in a preferred ratio of the two components. Thus, the mixture of HSA and therapeutic agent, added in the form of a solid, remains entrapped as a solid during the cross-linking reaction. Dextran, a polysaccharide, may be preferred over HSA as the carrier, since clinical grade dextran of about 70 kDa has a water solubility of about 30 mg/mL, which is >10-fold lower than HSA. Thus, the saturated dextran solution would be less viscous than the HSA solution.

Since the amount of solid therapeutic agent entrapped is above the solubility limit, then (under ideal conditions) as a given amount of the soluble agent is released from the depot, it is replenished by dissolution of solid therapeutic agent entrapped in the depot. As a result, the concentration of (soluble) therapeutic agent will remain constant, and hence the release rate will remain constant. In the example of quinine sulfate as a therapeutic agent, the water solubility is about 1 mg/mL and about 100 mg of solid quinine sulfate can be used to saturate the polymer solution and then be entrapped within 1 mL of gel. Thus, the highly desired zero order release kinetics should ensue as long as the solution remains saturated, as occurs while the initial 99 mg is being released. Then, only the final 1 mg (1%) of the agent will get released according to first order kinetics, since there is no more solid quinine to replenish the solution. During this tailing off period, the next sustained release dosage of therapeutic agent can be administered to the patient.

The possibility of administering the drug as a suspension of solid particles within a subcutaneous gel has an additional advantage with regard to drug loading. For many drugs, the combination of the water solubility of that drug and the amount needed for the duration of the sustained release period would require an unusually large volume of gel. For example, loading 50 mg of quinine sulfate at its solubility limit of 1 mg/mL would require a 50 mL depot. Besides the depot being unsightly, this could make the technology too expensive with regard to cost of polymer and cross-linker. Conversely, the duration of sustained release would have to be kept short to compensate for the limited loading capacity of a poorly water-soluble drug. Yet another consideration is the long-term chemical stability (weeks or months at body temperature) of the therapeutic agent; clearly, said therapeutic agent in most cases would be more stable as a solid rather than as an aqueous solution. Even though some or all of the therapeutic agent is administered as a solid, the present invention also comprises materials and methods for in situ formation of a gel matrix from a mixture containing said polymer(s) and said cross-linking reagent(s). Thus, the invention comprises both the gel and the solid particles of therapeutic agent. Depending on the desired repository site of the matrix of the invention, simply administering a therapeutic agent in the form of solid particles (microparticles, nanoparticles, etc.) could have undesirable attributes. These particles may migrate from the injection site or may be subject to attack by macrophages or soluble degradative enzymes or antibodies, in contrast with the protective environment afforded by a gel. Particles not contained within a gel would not be easily retrievable in case of an adverse side reaction, in contrast with the instant matrix. Furthermore in the present invention, the controlled release kinetics is supported by a small, well-defined gel compartment that can maintain the therapeutic agent as a saturated or near-saturated solution. Moreover, advantageous use of various excipients to maintain the stability of the active agents during residence in the instant compositions will permit the long-term use, and infrequent need to replace, the instant compositions.

The cross-linked matrix composition of the present invention may be provided in a form such as, but not limited to, a gel, microparticles, and nanoparticles. The composition may be processed for loading into capsules, for example, or for incorporation into another matrix or drug delivery system.

As mentioned above, release of an entrapped agent may be provided over the course of hours, days, or up to several months. In a further embodiment of the invention in which no release is desired, the compositions of the invention, in particular the cross-liked, emulsion-containing compositions, have their applicability in cosmetic surgery as long-lived, implantable materials to fill in or fill out particular sites in or on the body. In-situ formation of the implant by injection of the components before or during polymerization provides a non-surgical means for placing an inert, shapable mass at any site in the body. In a further embodiment, with the use of reducible cross-links as described above, such a cosmetic implant may be readily removed without surgery after it has achieved its desired purpose. One non-limiting example of such an application is in the theatre, where an actor may desire a temporary altered appearance, such as altered facial features, during the filming or a live production. Post-production, the implant can be depolymerized and flushed or allowed to be absorbed non-surgically.

In another aspect of the present invention, a method is provided for the controlled release of a therapeutic agent in an animal comprising administration to the animal a therapeutically effective amount of the therapeutic agent in one of the matrices described above. The matrix may contain more than one therapeutic agent, and an animal may be administered a single therapeutic agent in the form of more than one matrix, each with a particular controlled release kinetic profile.

Administration of the matrix of the present invention is performed to locate the matrix at a desired site for controlled delivery of the therapeutic agent. This may be to a particular body compartment to which the therapeutic agent has a desired targeted effect, or the matrix may be administered to a particular location wherein controlled release may provide the therapeutic agent for distribution throughout the body or to another site from which the administered site tow drains. Where a number of appropriate sites are possible, one may be selected from which the matrix may be easily removed. The particular site will be determined by the desired effect of the therapeutic agent.

Non-limiting examples of possible sites for administration of the matrix includes subcutaneous, oral, intravenous, intraperitoneal, intradermal, subdermal, intratumor, intraocular, intravisceral, intraglandular, intravaginal, intrasinus, intraventricular, intrathecal, intramuscular, and intrarectal. It will be seen that certain of these sites provides a site from which systemic distribution of the therapeutic agent may occur, for example, intraperitoneal, subcutaneous, and oral. Certain sites may be selected to provide a target tissue or organ to which the therapeutic agent's efficacy is desired, such as intratumor, intravaginal, intraglandular, intrathecal, intraventricular, and intraocular. For example, an antitumor agent may be entrapped in the matrix of the present invention and implanted in or near a tumor, for targeted delivery to the tumor.

A subject in whom administration of the pharmaceutical composition of the present invention is preferably a human, but can be any animal. Thus, as can be readily appreciated by one of ordinary skill in the art, the methods and pharmaceutical compositions of the present invention are particularly suited to administration to any animal, particularly a mammal, and including, but by no means limited to, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use. In addition, the composition of the present invention may also be used in non-medical situation where controlled release characteristics are desirable, such as, for example, controlled release of fertilizer or anti-parasite agents in the soil near plants; industrial settings, such as purification agents for drinking water tanks, etc. Thus, the term “therapeutic agent” is meant herein to refer to any agent desirous of controlled release.

The controlled release of the therapeutic agent from the matrix is believed to occur as a consequence of the diffusion from and/or biodegradation of the matrix by one or more in-vivo degradation pathways. While not wishing to be bound by theory, and by which the inventors herein have no duty to disclose or be bound, it is believed that degradation of the matrix is achieved by local factors at the site of administration such as reducing agents, for example, glutathione; reductases, S-transferases, peptidases, proteases, non-enzymatic hydrolysis, esterases and thioesterases. The varied presence of these various degradation agents in particular compartments in the body provides further guidance on selecting the appropriate site for administration, and also in the preparation of a matrix to provide the desired release kinetics in the presence of the particular degradative machinery at the site. Moreover, in compositions of the invention comprising a plurality of phases, the controlled release is further regulated by the presence of, and/or passage through, one or more phases of the composition. For example, as noted above, an insoluble agent in a solid phase may slowly dissolve in the aqueous or oil phase, and this soluble agent then passes out of the composition. A solid phase in the oil phase of an emulsion passes from the solid phase to the oil phase to the aqueous phase. By regulating the properties, relative amounts, presence of excipients, and other parameters of each of the phases, the release characteristics may be adjusted to provide the desired properties for the agent entrapped within the matrix of the compositions of the invention.

The controlled release characteristics of the pharmaceutical compositions of the invention may be selected for that suited to the particular use. In a preferred embodiment, zero order or pseudo zero order kinetics, i.e., constant release, is desired, where, for example, less than 3% of the therapeutic agent is released from the matrix during the first few hours, and then zero order release continues until at least about 80% of the therapeutic agent is released. First order release kinetics may also be provided.

In another embodiment of the present invention, the therapeutic agent in the above-described matrix is prepared immediately prior to or during administration to the animal. For example, just prior to administration, a solution, suspension or emulsion containing the therapeutic agent and the polymer can be mixed with a solution containing the cross-linking agent. Upon mixture, the cross-linking of the polymer begins to occur, entrapping the therapeutic agent. As cross-linking proceeds, the mixture changes from a liquid suspension to a gel. The immediately-mixed solutions can be administered as a liquid, for example, by subcutaneous injection, wherein the injected liquid continues to cross-link and change into a matrix at the site of administration. This simplifies the administration of a solid or semi-solid matrix. As the cross-linking traps the therapeutic agent, little is released in a burst during the process.

In yet another aspect, the present invention is directed to a pharmaceutical composition consisting of a matrix comprising a therapeutic agent exhibiting at least one first controlled release in-vivo kinetic profile, the matrix comprising at least one cross-linked polymer on which at least two thiol groups are present entrapping at least one therapeutic agent. In another embodiment, the therapeutic agent in the aforementioned matrix has at least one second controlled release in-vivo kinetic profile. Controlled release in vivo kinetic profiles refer to the particular release characteristics of the therapeutic agent from the matrix to provide therapeutically effective delivery of the therapeutic agent to the body.

Another aspect of the invention is the process by which the pharmaceutical compositions of the invention are prepared. The pharmaceutical composition is prepared by cross-linking a polymer on which functional groups are present and are capable of being cross-linked, such as having at least two thiol groups, by any one of various means, in the presence of the therapeutic agent to be entrapped. In a preferred embodiment, at least two thiol groups are present on the polymer. While the following discussion pertains in some instances to the use of thiol-containing polymers, it is understood that in accordance with the general discussions above, that other functional groups on the polymers may be used to achieve similar effects, and the discussions if not dependent on the particular properties of thiol-containing polymers are applicable generally to any and all compositions of the invention.

In yet a further aspect of the methods and pharmaceutical compositions of the present invention, the polymer or cross-linking agent may additionally comprise a functional group, such as an amino or carboxyl group. The functional group may be derivatized to provide on the polymer or cross-linking agent a moiety such as a label, for example, a contrast/imaging agent, a radionuclide, a chromophore, a fluorophore, a red or near-infrared fluorophore, or a nonradioactive isotope, such that the matrix may be readily located within the body, or the label may be used to monitor degradation of the matrix by detecting a metabolically stable moiety in the urine. The label may be chemically attached to the functional group by, for example, carbodiimide activation or use of a homobifunctional or heterobifunctional cross-linking agent. Examples of contrast/imaging agents include F-19 for MRI, I-126 for X-ray and Tc-99m for radioscintigraphy.

In a typical example of the preparation of a matrix of the invention, the first step is the synthesis of a polymer on which at least two thiol groups are present. In the case of the amide-linked polymer of α,ω-diamino-PEG with thiomalic acid, the thiomalic acid is first protected as (S-trityl)-thiomalic acid, as follows. Equimolar quantities of α,ω-diamino-PEG (MW 3,400, Shearwater Polymers) and (S-trityl)-thiomalic acid were dissolved in methylene chloride, and 3.5 equivalents of 1,3-diisopropylcarbodiimide (DIPC, Aldrich) was added to carry out a direct polycondensation at room temperature with 0.5 equivalent of 4-(dimethylamino)-pyridine (DMAP, Aldrich) and p-toluenesulfonic acid monohydrate (PTSA, Aldrich) as catalysts. The reaction mixture was precipitated with cold ethyl ether to obtain a white polymer product, which was treated with 100% trifluoroacetic acid for 2 hours to remove the protecting trityl groups from the polymer. The deprotected polymer was precipitated in cold ethyl ether, washed 5 times with ether and dried under vacuum. The molecular weight of the resulting PEG-thiomalic acid polymer was measured by size exclusion chromatography, using PEGs of defined molecular weights (Shearwater Polymers) for calibration of the column.

These polymers are then to be used for the preparation of the matrix entrapping the therapeutic agent in the presence of two or more phases. As mentioned above, use of polymers made from the smaller PEG subunits would result in a matrix having more closely spaced cross-links, resulting in a slower rate of diffusion of entrapped therapeutic agents, especially higher molecular weights, out of the matrix. Amide bonds, resulting from use of the diamino-PEGs, are expected to be considerably more stable in vivo than are ester bonds, which corresponds to a lower rate of degradation of the matrix in vivo.

For matrix formation, a preferred cross-linking reagent is α,ω-divinylsulfone-PEG (Shearwater Polymers). The vinylsulfone functional group reacts readily and specifically with thiol groups on the matrix-forming polymer, but will not react with disulfide bonds, such as present in a protein with disulfide bonds. As mentioned above, the possibility of cleavage of any disulfide bond in the therapeutic agent can be minimized or essentially prevented by providing steric hindrance to the thiol groups in the thiol-containing polymer.

Another factor influencing the release rate of the therapeutic agent is the size and the shape of the matrix depot. The greater the ratio of surface area to volume, the shorter the duration of release. For example, a sheet-like depot would be expected to release the encapsulated agent much faster than would a spherical depot of the same mass. One large sphere would release the agent more slowly than would many small spheres of the same total mass. The selection of the size and shape of the matrix will be readily determinable by a skilled artisan based on desired characteristics of release of the particular therapeutic agent. Other factors include the relative amount of each of the multiple phases present in the composition, the phase(s) in which the active agent or agents is or are present, the solubility of the agent(s) and partitioning between the phases, etc. By using the teaching herein, the skilled artisan can readily determine for a particular use and agent(s) the proper features of the desired composition and the means to prepare it.

As mentioned above, the matrix may be administered just after mixing the polymer on which at least two thiol groups are present with the cross-linking agent, in the presence of the therapeutic agent, in the one or more phases, such that the mixture may be injected in liquid form but the matrix solidifies into the cross-linked form soon thereafter. Fox example, to practice this aspect of the invention, a dual-syringe pump may be used for making and administering the mixture. For example, one syringe will be filled with 0.5 mL of matrix-forming polymer and the therapeutic agent in a plurality of phases, while the other syringe will be filled with 0.5 mL of the cross-linker solution (or the therapeutic agent may be mixed in this syringe), both at the optimal concentrations for the cross-linking reaction. The concentrations selected for these two solutions will be that appropriate to create the matrix with the appropriate controlled release kinetic profile. The pump will be set at a constant flow rate (e.g. 0.1 mL/min). The two solutions will be mixed in a tee-fitting and the mixture will be injected. The mixture becomes viscous as it flows through teflon tubing for a specified time. The mixed solution may be injected to the site of administration, whereupon the solution polymerizes into a multiphase hydrogel matrix. More simply, all components may be mixed just prior to administration.

More simply, all components can be mixed in one syringe just prior to administration. The rate at which the gel forms by the cross-linking reaction is preferably in a time frame of a few minutes. This rate may be controlled by the type of functional group on the cross-linking reagent and by the pH of the reaction, being slower at pH 6 compared with pH 7.

With regard to the administration of the matrix as described above, in one embodiment, may Add comprise, for example, several injections of 1 microliter each, perhaps repeated at multiple sites around the body, whereby the number and volume of the injections corresponds to a particular pharmacokinetic profile. As noted above, the fluid would be a partially cross-linked viscous matrix as it enters the skin, thereby already entrapping the drug. Microparticles, perhaps uniformly sized at 1 cubic millimeter (about 1 microliter), would harden within minutes as the cross-linking reaction goes to completion. Alternatively, a single needle injection may be used to produce a subcutaneous depot that may be easier to remove surgically in case of an adverse reaction to the depot or the drug.

Factors such as the size and shape of the matrix, the concentration and amount of the therapeutic agent entrapped therewithin, the extent of cross-linking of the polymer on which at least two thiol groups are present, the presence of certain excipients and the susceptibility of the polymer and cross-links to biodegradative machinery contribute to the pharmacokinetic profile of the therapeutic agent, the longevity of the matrix, among other factors. Each therapeutic agent will require a particular set of factors to provide the matrix with the correct profile for therapeutic use. In particular, the molecular weight and physical interaction between the agent and the polymers comprising the matrix will participate in the profile. For the practice of the invention, a particular set of preparation and operating conditions will be established for each therapeutic agent and, in more particular, the desired controlled release profile for that agent. It is well within the realm of the skilled artisan, based on the teaching herein, to determine the matrix components and other factors in the preparation of a suitable range of conditions for preparing a matrix for a particular therapeutic agent which exhibits the desired profile.

Further to the typical procedure described above for the preparation of the matrix of the present invention, variables for the protein solution include but are not limited to protein concentration, pH, salt content and presence of other excipients and stabilizers. The protein may be modified, such as by pegylation, to increase its size and, thereby, decrease its release rate.

In a further aspect of the present invention, a particular release rate may be achieved using a mixture of two or more starting polymer subunits to prepare the thiol-containing polymer or using a mixture of two or more polymers during the cross-linking/entrapment process. A delayed release product may be prepared by first entrapping the protein using an ester-type polymer, followed by coating or encapsulating these resulting particles using an amide-type polymer. The desired release kinetics for the final product may be achieved by administering to the patient a blend of two or more differently and separately cross-linked, entrapped protein preparations. Other means for making a product with a desired release profile will be apparent to the skilled artisan based on the teachings herein and should be considered to be within the scope and spirit of the present invention. As mentioned above, for any particular matrix, the release rate must be determined empirically in vivo, since it is dependent on many factors, including the size of the protein, diffusion from the matrix and the rate of degradation of the cross-linked polymer matrix due to the action of esterases, peptidases and reducing agents at the site of the depot.

The present invention may be better understood by reference to the following non-limiting Examples, which are provided as exemplary of the invention. The following examples are presented in order to more fully illustrate the preferred embodiments of the invention. They should in no way be construed, however, as limiting the broad scope of the invention.

### EXAMPLE 1

### Entrapment of Quinine Sulfate Monohydrate in a Thiol Containing Polymer Hydrogel Through a Suspension System

A thiol-containing polymer was prepared from thiomalic acid and α,ω-diamino-PEG as follows. One equivalent of thiomalic acid and 3 equivalents of trityl chloride were dissolved in dimethylformamide (DMF). The reaction was carried out at room temperature with stirring overnight. The reaction mixture was loaded onto a silica gel column and the eluted fractions containing trityl-thiomalic acid were collected and evaporated to dryness. Equimolar quantities of α,ω-diamino-PEG (MW 3,400; Shearwater Polymers, Inc. Huntsville, Ala.) and thiol group-protected thiomalic acid as prepared above were dissolved in methylene chloride, and 3.5 equivalents of 1,3-diisopropylcarbodiimide (DIPC, Aldrich, Milwaukee, Wis.) were added to carry out a direct polycondensation at room temperature with 0.5 equivalent of 4-(dimethylamino)-pyridine (DMAP, Aldrich, Milwaukee, Wis.) and p-toluenesulfonic acid monohydrate (PTSA, Aldrich, Milwaukee, Wis.) as catalyst. The reaction mixture was precipitated with cold ethyl ether to obtain a white polymer product. The polymer was treated with 100% trifluoroacetic acid (TFA) for 2 hours to remove the protecting trityl groups from the polymer pendant chain. The deprotected polymer was precipitated in cold ethyl ether, washed 5 times with ether and dried under vacuum.

Sixteen mg of the foregoing polymer was dissolved in 300 microliters of PBS, pH 7.4. Fifty mg of quinine sulfate monohydrate (Aldrich Chemical Co., Milwaukee, Wis.) was added into the polymer solution to form a suspension. Then, 4.7 mg PEG-(VS)₂ (MW 2000 Da, Shearwater Polymers, Inc., Huntsville, Ala.) was dissolved in 100 microliters of PBS, pH 7.4. The two solutions were mixed thoroughly in a 1.5 mL Eppendorf tube. The mixture was allowed to stand at room temperature (25 degree C.) until the hydrogel formed (“DepoGel formulation I”).

### EXAMPLE 2

### Entrapment of Quinine Sulfate Monohydrate in a Thiol Containing Polymer Hydrogel Through an Emulsion System

A thiol-containing polymer prepared from α,ω-dihydroxy-PEG and thiomalic acid was prepared as follows. Equimolar quantities of α,ω-dihydroxy-PEG and thiomalic acid were dissolved in methylene chloride, and 3.5 equivalent of 1,3-diisopropylcarbodiimide (DIPC, Aldrich, Milwaukee, Wis.) were added to carry out a direct polycondensation at room temperature with 0.5 equivalent of 4-(dimethylamino)-pyridine (DMAP, Aldrich, Milwaukee, Wis.) and p-toluenesulfonic acid monohydrate (PTSA, Aldrich, Milwaukee, Wis.) as catalyst. The reaction mixture was precipitated with cold ethyl ether to obtain a thiol-containing polymer.

Sixteen mg of the foregoing thiol-containing polymer was dissolved in 200 microliters of PBS, pH 7.4. To this, 200 microliters of ethyl myristate (Aldrich Chemical Co., Milwaukee, Wis.) was added as the oil phase and 24 mg of sodium dodecylsulfate (Bio-Rad, Hercules, Calif.) as the emulsifier. The mixture was mixed thoroughly to form an emulsion system. Fifty mg of quinine sulfate monohydrate (Aldrich Chemical Co., Milwaukee, Wis.) was added into the above emulsion system. Then, 4.7 mg PEG-(VS)₂ (MW 2000 Da, Shearwater Polymers, Inc., Huntsville, Ala.) was dissolved in 100 mL of PBS, pH 7.4. After thorough mixing in a 1.5 mL Eppendorf tube, the mixture was allowed to stand at room temperature (25 degree C.) until the hydrogel formed (“DepoGel formulation II”).

### EXAMPLE 3

### Release of Quinine Sulfate Monohydrate from Thiol Containing Polymer Hydrogels

To conduct a release study, 2 mL of PBS, pH 7.4 was added to the polymer hydrogel in a 5 mL test tube and allowed to incubate the hydrogel at room temperature for pre-selected time periods with rotation (about 100 rpm). The supernatant from the hydrogel was removed for fluorescence measurement and to add fresh PBS for the next incubation.

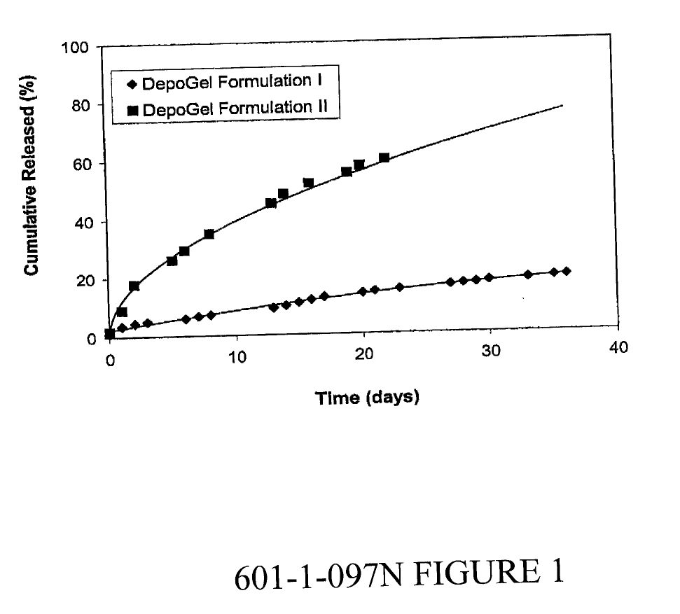

Fluorescence measurements of released quinine sulfate monohydrate were performed using FALCON microtiter plates from Becton Dickinson (Lincoln Park, N.J.) on a CytoFluorä II fluorescence multi-well plate reader (PerSeptive Biosystems, Framingham, Mass.). For each measurement, 100 microliters of release sample is mixed with 100 microliters of 1 M sulfuric acid in a well of the microtiter plate. An excitation wavelength of 360 nm and an emission wavelength of 460 nm are used for fluorescence measurements. Based on fluorescence measurement of each collected release sample, the release profiles of quinine sulfate monohydrate from thiol containing polymer hydrogels in PBS, pH 7.4 at 25 degree C. are shown in FIG. 1. DepoGel formulation I shows the quinine release from a thiol containing polymer hydrogel through a suspension system. DepoGel formulation II shows the quinine release from a thiol containing polymer hydrogel through a emulsion system.

### EXAMPLE 4

### Further Example of the In-Vitro Release of Quinine Sulfate

Single-phase system (R-gel): In a test tube, 16 mg of Thiol-PEG polymer were dissolved in 400 μl of PBS (pH=7.4), 50 mg of quinine sulfate was added to the polymer solution to form a suspension. In another test tube, 4.7 mg of PEG-divinylsulfone (PEGDVS) were dissolved in 100 μl of PBS (pH 7.4) as the cross-linker solution. The cross-linker solution is added into the above prepared suspension and it is mixed thoroughly. A polymer hydrogel is formed in about 3 minutes.

Two-phase (emulsion) system (E-gel): In a test tube, 16 mg of Thiol-PEG polymer and 24 mg SDS are dissolved in 200 μl of PBS (pH=7.4). 200 μl of ethyl myristate (“Oil”) are added and mixed thoroughly to form an emulsion. 50 mg of quinine sulfate is added to above emulsion. In another test tube, 4.7 mg of PEG-divinylsulfone (PEGDVS) are dissolved in 100 μl of PBS (pH 7.4) as the cross-linker solution. The cross-linker solution is added into the above prepared emulsion and mixed thoroughly. A polymer hydrogel is formed in about 3 minutes.

Release conditions and Sample collection: To each test tube containing Rgel and Egel, 2 mL of PBS is added. The test tubes are set on a rotational shaker (250 rpm) at room temperature (25° C.). At pre-selected time points, all solution is removed for sample analysis and 2 mL of fresh PBS is added into each tube.

Sample analysis: For each collected sample, 100 μl of sample is mixed with 100 μl of 1 M H₂SO₄ solution in a microplate. Fluorescence measurements were performed on a CytoFluor(tm) II fluorescence multi-well plate reader (PerSeptive Biosystems, Framingham, Mass.).

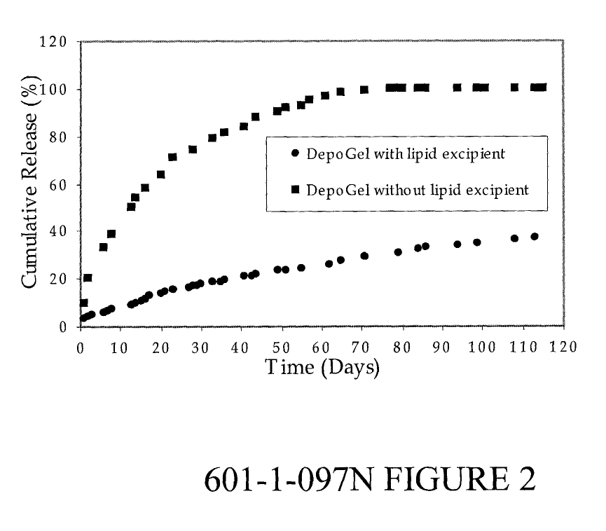

Results: FIG. 2 shows that the in-vitro release rate of a small molecule drug, quinine sulfate from E-gel (with a lipid excipient) which displays an apparent zero-order release profile and is much slower than release without excipient (R-gel). Only 40% of the quinine is released from the E-gel with excipient in 4 months of study, whereas essentially 100% is released without excipient in 2 months from the R-gel. There is no substantial initial burst effect.

### EXAMPLE 5

### In-vivo Release Study of Quinine Sulfate

Animal model: New Zealand great white rabbits were used for an in-vivo release study. The average weight of the rabbits was 3.0 kg. Three groups of rabbits were used for the study and each group contains 3 rabbits. Group A was used for subcutaneous injection of quinine sulfate solution (not in a polymer system). Group B was used for subcutaneous injection of quinine sulfate in Rgel, as described above. Group C was used for subcutaneous injection of quinine sulfate in Egel, the lipid emulsion described above.

Preparation of plain injection of quinine sulfate: For plain injection, 1 mL of 1 mg/mL quinine sulfate solution was injected subcutaneously into the upper back area of each rabbit in Group A.

Preparation of Rgel: For Rgel preparation, 16 mg of Thiol-PEG polymer is dissolved in 400 μl of PBS (pH=7.4), and 50 mg of quinine sulfate is added to the polymer solution to form a suspension. 4.7 mg of cross-linker, PEGDVS is dissolved in 100 μl of PBS (pH 7.4). The cross-linker solution is drawn into a 1 mL syringe first, then draw the thiol-PEG polymer solution into the same syringe. It was mixed thoroughly by drawing up and push down the syringe plunger several times. The solution gradually became viscous in 2 minutes; then this viscous solution was administered subcutaneously into the upper back area of each rabbit in Group B.

Preparation of Egel: For Egel preparation, 16 mg of Thiol-PEG polymer and 24 mg SDS were dissolved in 200 μl of PBS (pH=7.4). 200 μl of ethyl myristate (“Oil”) was added and mixed thoroughly to form an emulsion. 50 mg of quinine sulfate was added to above emulsion. In another test tube, 4.7 mg of cross-linker, PEGDVS was dissolved in 100 μl of PBS (pH 7.4). The cross-linker solution is drawn into a 1 mL syringe, followed by the thiol-PEG polymer solution. The syringe contents were mixed thoroughly by drawing the syringe plunger up and down several times. The solution gradually became viscous in 2 minutes; the viscous solution was administered subcutaneously into the upper back area of each rabbit in Group C.

Sample collection: At pre-selected time points, 2 mL of blood was collected from vein of the rabbit ear into an EDTA-treated test tube. The blood was centrifuged at 3000×g at 4° C. to obtain about 1 mL of plasma. All plasma samples were kept at −70° C. until analysis.

Sample analysis: Reverse-phase HPLC method was used for plasma sample analysis under following conditions: HPLC column: Princeton SPHER ULTIMA C18 100 Å 5μ 150×4.6 nm Mobile Phase: 95/5 25 mM KH₂PO₄, pH3/Methanol. Flow rate: 1 mL/min

Sample treatment: Plasma was precipitated with 2 volumes of cold methanol, vortexed and centrifuges at 1500×g for 10 min. The supernatant (10 μl) was injected into the HPLC column.

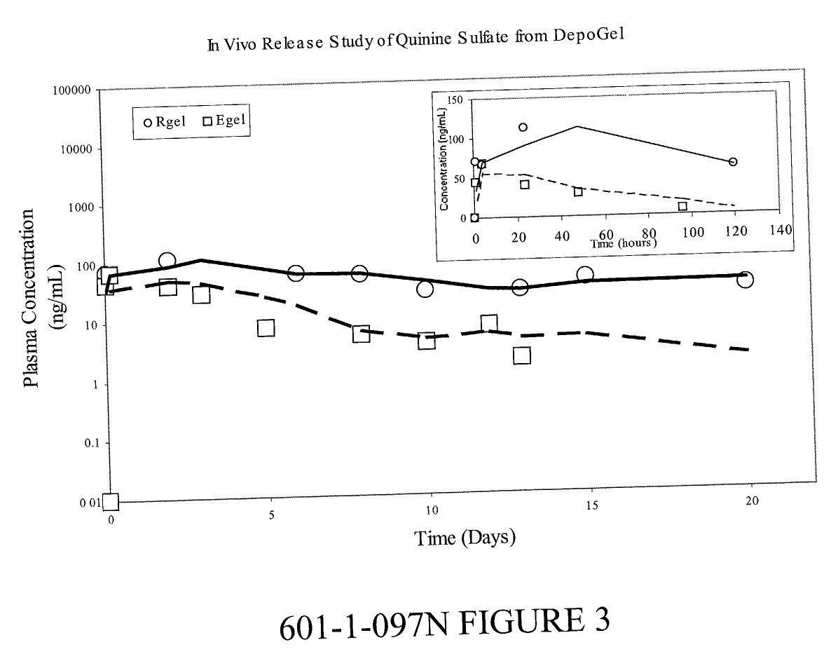

Results: FIG. 3 shows the in-vivo release of a small molecule drug, quinine sulfate from polymer systems of the present invention in rabbits. Egel containing a lipid excipient displays a slower drug release than that of Rgel, the polymer system without excipient. There are no substantial initial burst effects in either case.

### EXAMPLE 6

### In-vitro Release Study of Salmon Calcitonin

Preparation of polymer system without lipid excipient (Rgel): In a series of test tubes, 20 mg of soluble polymer was dissolved in PBS (pH 5.5) to yield 400 μl of solution in each tube. In another series of test tubes, 1 mg of cross-linker and 1 mg of salmon calcitonin were dissolved in 100 μl of PBS (pH 5.5). The two solutions were mixed thoroughly at room temperature (25° C.), and a series of polymer hydrogels was formed in about 1 min.

Preparation of polymer system containing lipid excipient (Egel): In a series of test tubes, 20 mg of soluble polymer and varying amounts of lipid excipient were dissolved in PBS (pH 5.5) to yield 400 μl of solution in each tube. In another series of test tubes, 1 mg of cross-linker and 1 mg of salmon calcitonin were dissolved in 100 μl of PBS (pH 5.5). The two solutions were mixed thoroughly at room temperature (25° C.), and a series of polymer hydrogels was formed in about 1 min.

Release Conditions and Sample Collection: To each test tube containing a polymer system, 1 mL of PBS (pH 5.5) was added. The test tubes were set on a rotational shaker (300 rpm) at room temperature (25° C.). At pre-selected time points, all solution was removed from each tube for sample analysis and 1 mL of fresh PBS (pH 5.5) was added to each tube.

Sample analysis: All collected samples are analyzed by HPLC as described above.

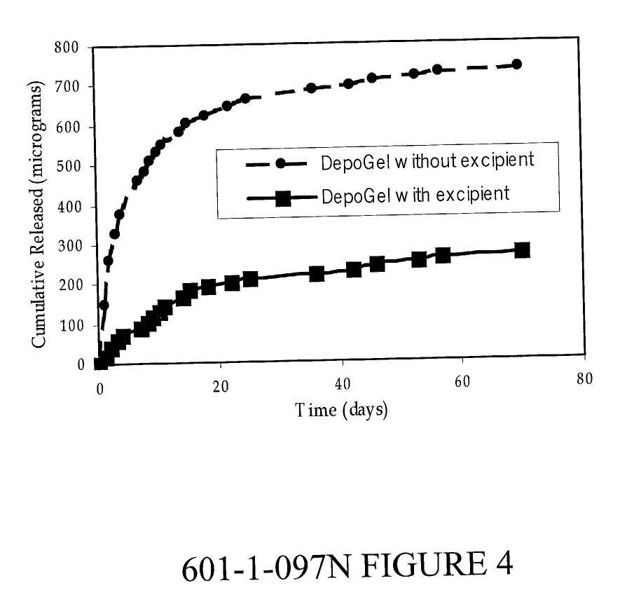

Results: FIG. 4 shows that the use of the lipid excipient-containing cross-linked polymer significantly slowed the in-vitro release rate of a peptide drug, salmon calcitonin (sCT) from the polymer.

### EXAMPLE 7

### In-vivo Release Study of Salmon Calcitonin

Animal model: New Zealand great white rabbits were used for an in-vivo release study. The average weight of the rabbits was 3.0 kg. Two groups of rabbits were used for the study and each group contained 3 rabbits. Group A was used for subcutaneous injection of salmon calcitonin in Rgel. Group B was used for subcutaneous injection of salmon calcitonin in Egel.

Preparation of Rgel (also referred to herein as Depogel Formulation I): For Rgel preparation, 40 mg of Thiol-PEG polymer is dissolved in 800 μl of PBS (pH=7.4). 10 mg of salmon calcitonin and 2 mg of the cross-linker 1,11-bis-maleimidotetraethylene glycol [BM(EG)₄] were dissolved in 200 μl of PBS (pH 7.4). The cross-linker solution was drawn into a 3 mL syringe first, then the thiol-PEG polymer solution was drawn into the same syringe. It was mixed thoroughly by drawing up and pushing down the syringe plunger several times. The solution gradually became viscous within 1 minute; then this viscous solution is administered subcutaneously into the upper back area of each rabbit in Group A. A soft, round-shaped depot is formed at the injection site upon injection.

Preparation of Egel (also referred to herein as Depogel Formulation II): For Egel preparation, 40 mg of Thiol-PEG polymer and 5 mg SDS are dissolved in 700 μl of PBS (pH=7.4). 100 μl of ethyl myristate (“Oil”) was added and mixed thoroughly to form an emulsion. In another test tube, 10 mg of salmon calcitonin and 2 mg of cross-linker, BM(EG)₄ were dissolved in 200 μl of PBS (pH 7.4). The cross-linker solution was drawn into a 3 mL syringe, followed by the thiol-PEG polymer solution containing oil droplets (i.e. an emulsion system). The syringe contents were mixed thoroughly by drawing the syringe plunger up and down several times. The solution gradually became viscous within 1 minute; the viscous solution was administered subcutaneously into the upper back area of each rabbit in Group B. A soft, round-shaped depot was formed at the injection site upon injection.

Sample collection: At pre-selected time points, 1 mL of blood was collected from vein of the rabbit ear into a heparin-treated test tube. The blood was centrifuged at 3000 g at 4° C. to obtain about 0.5 mL of plasma. All plasma samples were kept at −70° C. until analysis.

Sample analysis: A radioimmunoassay (RIA) was used for plasma sample analysis to determine the salmon calcitonin level in rabbit plasma.

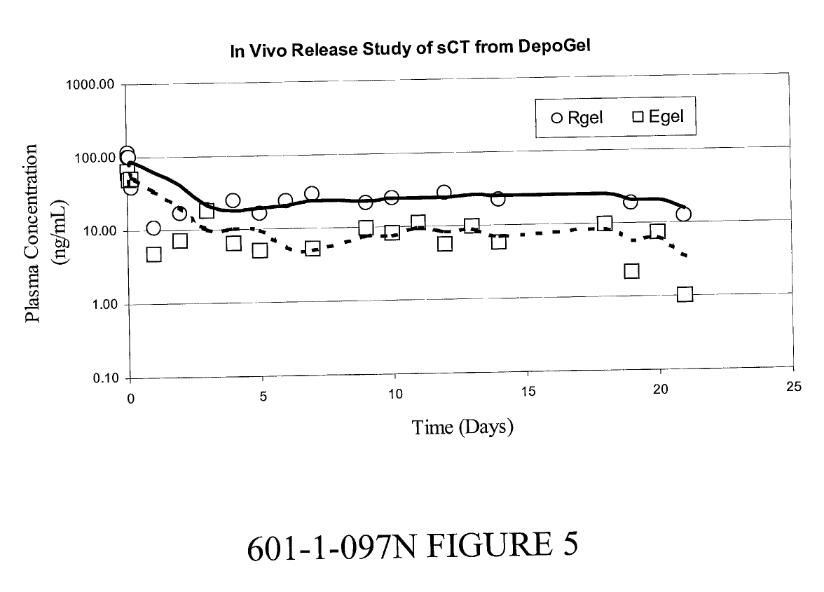

Results: FIG. 5 shows the in-vivo release of a peptide drug, salmon calcitonin, from polymer systems of the invention in rabbits. Egel, containing a lipid excipient, displays a slower drug release than that of Rgel, without excipient. There are no substantial initial burst effects in either case.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Various publications are cited herein, the disclosures of which are incorporated by reference in their entireties.

What is claimed is:

1. A pharmaceutical composition comprising a matrix capable of delivering at least one therapeutic agent to a bodily compartment under controlled release conditions, said matrix comprising a homogeneous mixture of aqueous phase and at least one other phase, at least one therapeutic agent present in at least one of said phases, and at least one cross-linked polymer physically entrapping said at least one therapeutic agent.

2. The pharmaceutical composition of claim 1 wherein said at least one other phase is a solid phase, an oil phase, or a combination thereof.

3. The pharmaceutical composition of claim 2 wherein said oil phase and said aqueous phase are in the form of an emulsion.

4. The pharmaceutical composition of claim 1 wherein said polymer comprises a backbone selected from the group consisting of a poly(alkylene oxide), carboxymethylcellulose, dextran, modified dextran, polyvinyl alcohol, N-(2-hydroxypropyl)methacrylamide, polyvinyl pyrrolidone, poly-1,3-dioxolane, poly-1,3,6-trioxane, polypropylene oxide, a copolymer of ethylene and maleic anhydride, a polylactide/polyglycolide copolymer, a polyaminoacid, a copolymer of poly(ethylene glycol) and an amino acid, and a polypropylene oxide/ethylene oxide copolymer.

5. The pharmaceutical composition of claim 1 wherein said polymer comprises at least two functional or reactive groups.

6. The pharmaceutical composition of claim 5 wherein said functional groups are amino, carboxyl, thiol, hydroxy, or any combination thereof.

7. The pharmaceutical composition of claim 6 wherein said polymer is an poly(alkylene oxide) derivative.

8. The pharmaceutical composition of claim 7 wherein said poly(alkylene oxide) derivative is selected from the group consisting of α,ω-dihydroxy-poly(ethylene glycol) and α,ω-diamino-poly(ethylene glycol).

9. The pharmaceutical composition of claim 6 wherein said functional groups are thiol groups.

10. The pharmaceutical composition of claim 9 wherein said polymer is prepared from α,ω-diamino-poly(ethylene glycol) and thiomalic acid; α,ω-dihydroxy-poly(ethylene glycol) and thiomalic acid; or α,ω-dicarboxy-PEG subunits and lysine, wherein free carboxy groups on said lysine are derivatized to provide thiol groups.

11. The pharmaceutical composition of claim 9 wherein said thiol groups on said polymer are cross-linked by thioether or disulfide bonds.

12. The pharmaceutical composition of claim 9 wherein said thiol groups on said polymer are sterically hindered.

13. The pharmaceutical composition of claim 1 wherein said at least one therapeutic agent is selected from the group consisting of a small-molecule drug, a protein, a nucleic acid and a polysaccharide.

14. The pharmaceutical composition of claim 13 wherein said small-molecule drug is selected from the group consisting of an anticancer drug, a cardiovascular drug, an antibiotic, an antifungal, an antiviral drug, an AIDS drug, an HIV-1 protease inhibitor, a reverse transcriptase inhibitor, an antinociceptive drug, a hormone, a vitamin, an anti-inflammatory drug, an angiogenesis drug, and an anti-angiogenesis drug.

15. The pharmaceutical composition of claim 1 wherein said matrix has at least one controlled release in-vivo kinetic profile selected from the group consisting of zero order, pseudo zero order, and first order.

16. The pharmaceutical composition of claim 1 wherein said controlled release conditions is a constant rate of release.

17. The pharmaceutical composition of claim 1 wherein said matrix further comprises an excipient.

18. The pharmaceutical composition of claim 17 wherein said excipient is selected from the group consisting of a monovalent metal ion, a polyvalent metal ion, an anionic polymer, a cationic polymer, a nonionic polymer, a surfactant, and a protein.

19. A method for preparing the pharmaceutical composition of claim 1 comprising the steps of
i) preparing a mixture comprising at least one therapeutic agent and at least two phases one of which is an aqueous phase, said aqueous phase comprising a polymer having at least two functional groups thereon;
ii) cross-linking said polymer under conditions to form a cross-linked matrix having said therapeutic agent entrapped therein.

20. The method of claim 19 wherein said functional groups are thiol groups.

21. The method of claim 20 wherein said conditions that cause cross-linking of said thiol groups comprises reaction in the presence of an oxidizing agent or reaction with a cross-linking agent.

22. The method of claim 21 wherein said oxidizing agent is selected from the group consisting of molecular oxygen, hydrogen peroxide, dimethylsulfoxide, and molecular iodine.

23. The method of claim 21 wherein said cross-linking agent is a bifunctional disulfide-forming or thioether-forming cross-linking agent.

24. The method of claim 23 wherein said cross-linking agent is selected from the group consisting of 1,4-di-[3′,2′-pyridyldithio(propionamido)butane]; α,ω-di-O-pyridyldisulfidyl-poly(ethylene glycol); α,ω-divinylsulfone-poly(ethylene glycol); α,ω-diiodoacetamide-poly(ethylene glycol) and 1,11-bis-maleimidotetraethylene glycol.

25. A method for delivering at least one therapeutic agent to a bodily compartment to an animal under controlled release conditions comprising providing in said bodily compartment a pharmaceutical composition set forth in claim 1.

26. The method of claim 25 wherein said bodily compartment is subcutaneous, oral, intravenous, intraperitoneal, intradermal, subdermnal, intratumor, intraocular, intravisceral, intraglandular, intravaginal, intrasinus, intraventricular, intrathecal, intramuscular, and intrarectal.

27. The method of claim 26 wherein said composition is provided to said bodily compartment by a route selected from the group consisting of subcutaneous, oral, intravenous, intraperitoneal, intradermal, subdermal, intratumor, intraocular, intravisceral, intraglandular, intravaginal, intrasinus, intraventricular, intrathecal, intramuscular, and intrarectal.

28. The method of claim 25 wherein said controlled release conditions occur as a consequence of diffusion from said matrix or biodegradation of said matrix by an in-vivo degradation pathway selected from the group consisting of reducing agents, reductases, S-transferases, peptidases, proteases, non-enzymatic hydrolysis, esterases and thioesterases.

29. The method of claim 25 wherein said providing in said bodily compartment is carried out by forming said matrix immediately prior to or during administration of said matrix to said animal.

30. The method of claim 29 wherein said pharmaceutical composition is capable of being injected as a liquid or semisolid gel through a small gauge needle, begins cross-linking and entrapping said therapeutic agent during injection, and completes cross-linking and physically entrapping said therapeutic agent within several minutes of being injected.

31. The method of claim 25, wherein said controlled release conditions comprise a time course of release of five or more days.

32. The method of claim 31, wherein said time course of release is twenty or more days.

33. The method of claim 25, wherein said releasing comprises a controlled release profile comprising an optional bolus release profile followed by a release profile selected from the group consisting of zero order, pseudo zero order, and first order.

34. A method of administering a controlled release therapeutic agent to a mammal, said method comprising: preparing a solution comprising a hydrogel forming polymer having two or more thiol groups and a plurality of phases, one of which is an aqueous phase, a cross-linking agent comprising two or more thiol-reactive groups, and a therapeutically effective amount of drug; and injecting said mammal with said solution whereby a hydrogel drug depot is formed at the site of injection having said drug temporarily entrapped therein.

35. The method of claim 34, wherein said controlled release therapeutic agent has a kinetic profile comprising an optional initial bolus release profile followed by a release profile selected from the group consisting of zero order, pseudo zero order, and first order.

36. The method of claim 34 wherein said thiol-reactive cross-linking agent is an oxidizing agent; 1,4-di-[3′,2′-pyridyldithio(propionamido)butane]; α,ω-di-O-pyridyldisulfidyl-poly(ethylene glycol); α,ω-divinylsulfone-poly(ethylene glycol); α,ω-diiodoacetamide-poly(ethylene glycol) or 1,11-bis-maleimidotetraethylene glycol.

37. A hydrogel composition comprising a homogeneous mixture of aqueous phase and at least one other phase, and at least one cross-linked polymer in one of said phases.
